(19) United States
(12) Patent Application Publication
(10) Pub. No.: US 2010/0044918 A1
(43) Pub. Date: Feb. 25, 2010

(54) METHOD OF PREPARING SOLID REAGENT AND MICROFLUIDIC DEVICE EMPLOYING THE SOLID REAGENT

(75) Inventors: Yangui Lee, Seoul (KR); Mijeong Song, Suwon-si (KR); Jaechan Park, Yongin-si (KR); Yoonkyoung Cho, Suwon-si (KR); Jeonggun Lee, Seoul (KR)

Correspondence Address: SUGHRUE MION, PLLC, 2100 PENNSYLVANIA AVENUE, N.W., SUITE 800, WASHINGTON, DC, 20037, US

(73) Assignee: Samsung Electronics Co., Ltd., Suwon-si (KR)

(21) Appl. No.: 12/475,699

(22) Filed: Jun. 1, 2009

(30) Foreign Application Priority Data
Aug. 22, 2008 (KR) 10-2008-0082368

(51) Int. Cl. B29C 39/00; G01N 33/00

(52) U.S. Cl. 264/297.8; 422/68.1

(57) ABSTRACT

In a method of preparing a solid reagent, a liquid reagent is loaded into a plurality of reagent cavities formed in a mold, the loaded liquid reagent is frozen, the frozen reagent is separated from the mold, and the separated frozen reagent is dried to remove humidity therein.

### CROSS-REFERENCE TO RELATED APPLICATIONS

This application claims the benefit of Korean Patent Application No. 10-2008-0082368, filed on Aug. 22, 2008, in the Korean Intellectual Property Office, and the disclosure of which is incorporated herein in their entirety by reference.

### BACKGROUND

1. Field

One or more embodiments of the present invention relate to a method of preparing a solid reagent using a liquid reagent and a microfluidic device employing the solid reagent.

2. Description of the Related Art

Various methods of analyzing a sample have been developed to, for example, monitor environments, examine food, or diagnose the medical condition of a patient. However, these methods require many manual operations and the use of various devices. To perform an assay or test according to a predetermined protocol, an operator repeatedly performs various manual operations such as loading a reagent, mixing, isolating and transporting, reacting, and centrifuging. However, such repeated manual operations often result in erroneous results.

To perform tests quickly, skilled clinical pathologists are needed. However, it is hard even for a skilled clinical pathologist to perform various tests simultaneously. Also, quick results are necessary for immediate treatment of emergency patients. Accordingly, there is a need to develop various types of equipment enabling the simultaneous, rapid, and accurate tests for given circumstances.

Conventional pathological tests are performed with large and expensive pieces of automated equipment and a relatively large amount of a sample, such as blood. Moreover, results of pathological tests are only available 2 days (at the minimum) to roughly 2 weeks after receiving the blood sample from a patient.

In order to solve the above described problems, small and automated pieces of equipment for analyzing a sample taken from one patient or, if necessary, samples taken from a small number of patients over a short time period have been developed. In an example of such a system, blood is loaded into a disk-shaped microfluidic device and the disk-shaped microfluidic device is rotated, and then serum can be isolated from blood due to the centrifugal force. The isolated serum is mixed with a predetermined amount of a diluent and the mixture then flows into a plurality of reaction chambers in the disk-shaped microfluidic device. The reaction chambers are filled with reagents prior to allowing the mixture to flow therein. Reagents used may differ according to various goals of the blood tests. When the serum reacts with different reagents, predetermined colors may appear, and the change in color is used to perform blood analysis.

However, storing a reagent in a liquid state is difficult. U.S. Pat. No. 5,776,563 discloses a system in which a lyophilized reagent is stored and, when blood analysis is performed, the required amount of the lyophilized reagent is loaded into reaction chambers of a disk-shaped microfluidic device.

### SUMMARY

One or more embodiments are a method of preparing a solid reagent from a liquid reagent and a microfluidic device employing the solid reagent.

Additional aspects and/or advantages will be set forth in part in the description which follows and, in part, will be apparent from the description, or may be learned by practice of the invention.

One or more embodiments of the present invention may include: a method of preparing a solid reagent, the method including: preparing a mold having a plurality of reagent cavities; loading a liquid reagent to the plurality of reagent cavities; freezing the liquid reagent; separating the frozen reagent from the mold; and drying the frozen reagent to remove humidity contained therein.

According to an embodiment of the present inventive concept, the drying may include sublimating humidity contained in the frozen reagent.

According to an embodiment of the present inventive concept, the liquid reagent is concentrated to a level equal to or higher than that required for examination and loaded into the plurality of reagent cavities.

According to an embodiment of the present inventive concept, the plurality of reagent cavities may accommodate at least two different liquid reagents.

According to an embodiment of the present inventive concept, the plurality of reagent cavities may have at least two different internal configurations.

According to an embodiment of the present inventive concept, the mold may be flexible.

According to an embodiment of the present inventive concept, the solid reagent may be selected from the group consisting of reagents for detecting serum, aspartate aminotransferase (AST), albumin (ALB), alkaline phosphatase (ALP), alkaline aminotransferase (ALT), amylase (AMY), urea nitrogen (BUN), calcium (Ca⁺⁺), total cholesterol (CHOL), creatine kinase (CK), chloride (Cl⁻), creatinine (CREA), direct bilirubin (D-BIL), gamma glutamyl transferase (GGT), glucose (GLU), high-density lipoprotein cholesterol (HDL), potassium (K⁺), lactate dehydrogenase (LDH), low-density lipoprotein cholesterol (LDL), magnesium (Mg), phosphorus (PHOS), sodium (Na⁺), total carbon dioxide (TCO₂), total bilirubin (T-BIL), triglycerides (TRIG), uric acid (UA), albumin (ALB), and total protein (TP).

The liquid reagent may include a filler. According to an embodiment of the present inventive concept, the filler may be selected from the group consisting of bovine serum albumin (BSA), polyethylene glycol (PEG), dextran, mannitol, polyalcohol, myo-inositol, citric acid, ethylene diamine tetraacetic acid disodium salt (EDTA2Na), and polyoxyethylene glycol dodecyl ether (BRIJ-35).

According to an embodiment of the present inventive concept, the solid reagent may include a surfactant. According to an embodiment of the present inventive concept, the surfactant may be selected from the group consisting of polyoxyethylene, lauryl ether, octoxynol, polyethylene alkyl alcohol, nonylphenol polyethylene glycol ether; ethylene oxide, ethoxylated tridecyl alcohol, polyoxyethylene nonylphenyl ether phosphate sodium salt, and sodium dodecyl sulfate.

One or more embodiments include a microfluidic device including: a sample chamber to accommodate a sample to be tested; a diluent chamber to accommodate a diluent; a reagent chamber to accommodate a solid reagent; and a channel connecting the sample chamber, the diluent chamber, and the reagent chamber.

According to an embodiment of the present inventive concept, the microfluidic device may further include a valve controlling flow of a fluid through the channel. According to an embodiment of the present inventive concept, the valve may be formed of a valve forming material that melts by electromagnetic radiation energy. According to an embodiment of the present inventive concept, the valve forming material may be selected from a phase transition material and a thermoplastic resin, wherein the phase of the phase transition material or thermoplastic resin changes by electromagnetic radiation energy. According to an embodiment of the present inventive concept, the valve forming material may include micro heat-dissipating particles which are dispersed in the phase transition material, and absorb the electromagnetic radiation energy and dissipate the energy.

One or more embodiments of the present invention include a microfluidic device including: a platform having a plurality of chambers; and a solid reagent accommodated in at least one of the plurality of chambers, wherein the solid reagent has an accurate amount.

According to an embodiment of the present inventive concept, the plurality of chambers may include: a sample chamber to accommodate a sample to be tested; a diluent chamber to accommodate a diluent; and a plurality of reagent chambers to accommodate a solid reagent.

According to an embodiment of the present inventive concept, at least two different solid reagents are accommodated in the plurality of reagent chambers, wherein the different solid reagents have different shapes. According to an embodiment of the present inventive concept, the microfluidic device may further include: a channel connecting the plurality of chambers; and a valve placed in the channel to control flow of the fluid through the channel, wherein the valve blocks the channel when it is in a solid state, and the valve is melted by electromagnetic radiation energy to open the channel.

### BRIEF DESCRIPTION OF THE DRAWINGS

These and/or other aspects and advantages will become apparent and more readily appreciated from the following description of the embodiments, taken in conjunction with the accompanying drawings of which:

FIG. 1 is a plan view of a microfluidic device according to an embodiment of the present inventive concept;

FIG. 2 is a cross-sectional view of the microfluidic device of FIG. 1, wherein the microfluidic device is a two-layered microfluidic device according to an embodiment of the present inventive concept;

FIG. 3 is a cross-sectional view of the microfluidic device of FIG. 1, wherein the microfluidic device is a three-layered microfluidic device according to another embodiment of the present inventive concept;

FIGS. 4A through 4C are views illustrating a method of preparing a solid reagent according to an embodiment of the present inventive concept;

FIG. 5 is a sectional view of a channel that is opened by a valve;

FIG. 6 is a schematic view of an analyzer using the microfluidic device of FIG. 1;

FIG. 7 is a plan view of a microfluidic device including a disk-type platform according to another embodiment of the present inventive concept;

FIG. 8 is a plan view of an example of a modification of the microfluidic device of FIG. 7;

FIG. 9 is a plan view of a microfluidic device including a centrifuging unit according to another embodiment of the present inventive concept;

FIG. 10 is a view to explain a detection operation including 2-step reactions using the microfluidic device of FIG. 9;

FIG. 11 is a plan view of a microfluidic device including a container for loading a diluent according to another embodiment of the present inventive concept; and

FIGS. 12A and 12B are sectional views of the microfluidic device of FIG. 11.

### DETAILED DESCRIPTION

Reference will now be made in detail to embodiments, examples of which are illustrated in the accompanying drawings, wherein like reference numerals refer to the like elements throughout. In this regard, the present invention may be embodied in many different forms and should not be construed as being limited to the embodiments set forth herein. Accordingly, embodiments are merely described below, by referring to the figures, to explain aspects of the present invention.

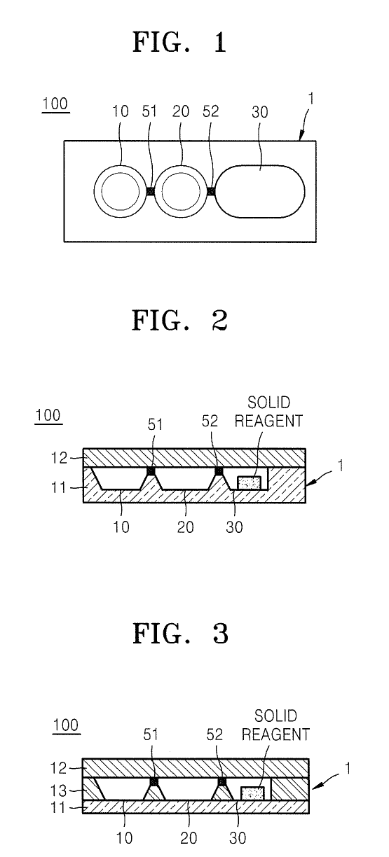

FIG. 1 is a plan view of a microfluidic device 100 according to an embodiment of the present inventive concept, and FIGS. 2 and 3 are cross-sectional views of the microfluidic device 100 of FIG. 1, according to two different embodiments of the present inventive concept. Referring to FIGS. 1 and 2, the microfluidic device 100 has a platform 1 having a microfluidic structure including a portion for storing a fluid and a channel through which the fluid flows. The platform 1 may be formed of a plastic material that can be easily molded and is biologically inactive. Examples of the plastic material include acryl, polymethyl methacrylate (PMMA), and a cyclic olefin copolymer (COC). However, a material for forming the platform 1 is not limited to those materials described above and can be any material that has chemical and biological stability, optical transparency, and mechanical processibility. The platform 1 may have, as illustrated in FIG. 2, a two-layer structure including a bottom (first) plate 11 and a top (second) plate 12. The platform 1 can also have, as illustrated in FIG. 3, a three-layered structure including a bottom (first) plate 11, a top (second) plate 12, and a partitioning (or intermediate) plate 13 disposed between the bottom plate 11 and the top plate 12. The partitioning plate 13 defines a portion for storing a fluid and a channel through which the fluid flows. The bottom plate 11, the top plate 12, and the partitioning plate 13 can be bonded together by using various materials such as double-sided tape or an adhesive, or by fusing supersonic waves. The platform 1 can also have other structures.

Hereinafter, a microfluidic structure for a blood test formed in the platform 1 will be described in detail. A sample chamber 10 is formed in the platform 1. The sample chamber 10 contains a sample, such as blood or serum. A diluent chamber 20 contains a diluent that is used to dilute the sample to a desired concentration suitable for tests. The diluent may be, for example, a buffer or distilled water (DI). A reagent chamber 30 contains a reagent for detecting a target material in the sample.

The sample chamber 10 is connected to the diluent chamber 20. The diluent chamber 20 is connected to the reagent chamber 30. A valve 51 is located between the sample chamber 10 and the diluent chamber 20 to control flow of a fluid between the sample chamber 10 and the diluent chamber 20. A valve 52 is located between the diluent sample 20 and the reagent chamber 30 to control flow of a fluid between the diluent sample 20 and the reagent chamber 30. Although not illustrated, the platform 1 may include: inlets for loading the sample, the diluent, and the reagent; and an air vent for discharging air.

In a sample analysis process which will be described later, light is projected into the reagent chamber 30. Thus, at least a portion of the platform 1 in which the reagent chamber 30 is located may be formed of a material that transmits light.

Various types of microfluidic valves can be used as the valves 51 and 52. For example, the valves 51 and 52 may be valves that open or close according to a flow rate of the fluid in the microfluidic structure, that is, valves that passively open when applied pressure that is generated due to flow of the fluid reaches or exceeds a predetermined level. Examples of such valves include a capillary valve having a micro channel structure, a siphon valve, and a hydrophobic valve which has a surface treated with a hydrophobic material. Such valves may be controlled according to a rotation rate of a microfluidic device. That is, as the rotation rate of the microfluidic device is increased, more pressure is applied to a fluid in the microfluidic device, and if the applied pressure reaches or exceeds a predetermined level, the valves open and the fluid flows.

In addition, the valves 51 and 52 can also be valves that are actively operated when an operation signal is transmitted and an operating power is externally provided. In the current embodiment, the valves 51 and 52 are values that operate when a valve forming material absorbs electromagnetic radiation emitted from an external source. The valves 51 and 52 are so called “normally closed” valves that block the flow of the fluid before electromagnetic radiation energy is absorbed.

The valve forming material may be a thermoplastic resin, such as a cyclic olefin copolymer (COC), polymethylmethacrylate (PMMA), polycarbonate (PC), polystyrene (PS), polyoxymethylene (POM), perfluoralkoxy (PFA), polyvinylchloride (PVC), polypropylene (PP), polyethylene terephthalate (PET), polyetheretherketone (PEEK), polyamide (PA), polysulfone (PSU), or polyvinylidene fluoride (PVDF).

The valve forming material can also be a phase transition material that exists in a solid state at room temperature. The phase transition material is loaded when in a liquid state into channels, and then solidified to close the channels. The phase transition material may be wax. When heated, wax melts into a liquid and the volume thereof increases. The wax may be, for example, paraffin wax, microcrystalline wax, synthetic wax, or natural wax. The phase transition material may be gel or a thermoplastic resin. Examples of the gel may include polyacrylamides, polyacrylates, polymethacrylates, and polyvinylamides.

In the phase transition material, a plurality of micro heat-dissipating particles that absorb electromagnetic radiation energy and dissipate thermal energy may be dispersed. The diameter of the micro heat-dissipating particles may be 1 nm to 100 μm so that the micro heat-dissipating particles freely pass through micro fluid channels having a depth of 0.1 mm and a width of 1 mm. When electromagnetic radiation energy of, for example, a laser ray, is supplied, the temperature of the micro heat-dissipating particles increases significantly, and thus, the micro heat-dissipating particles dissipate thermal energy and become uniformly dispersed in the wax. Individual micro heat-dissipating particle having the characteristics described above includes a core including metal and a hydrophobic shell. For example, the individual micro heat-dissipating particle may include a core formed of Fe, and a plurality of surfactants that are bonded to and cover the Fe. The micro heat-dissipating particles may be stored in a state of being dispersed in carrier oil. The carrier oil may be hydrophobic to uniformly disperse micro heat-dissipating particles having a hydrophobic surface structure. The carrier oil in which the micro heat-dissipating particles are dispersed is mixed with a molten phase transition material, and the obtained mixture is loaded between chambers and solidified, thereby blocking the flow of the fluid between the chambers.

The micro heat-dissipating particles may be, in addition to the polymer particles described above, quantum dots or magnetic beads. The micro heat-dissipating particles can also be micro particles of metal oxide, such as Al₂O₃, TiO₂, Ta₂O₃, Fe₂O₃, Fe₃O₄, or HfO₂. However, the inclusion of the micro heat-dissipating particles in the valves 51 and 52 is optional. For example, the valves 51 and 52 can be formed of only a phase transition material. A portion of the platform 1 corresponding to the valves 51 and 52 may be transparent to electromagnetic radiation irradiated from an external source so that the electromagnetic radiation is incident on the valves 51 and 52.

Since preserving a reagent in a liquid state is difficult, the reagent is formed into a solid state for storage until the use of the microfluidic device. For example, a liquid reagent is loaded to a mold having a selected shape and lyophilized, thereby forming reagent particles (e.g. beads) having a substantially uniform size, and then, according to the amount of a reagent desired or suitable for the test, an appropriate number of reagent beads are loaded into the reagent chamber 30. However, the lyophilized solid reagent can be broken when separated from the mold, and thus, it may not be necessarily easy to manufacture a substantially uniform size of reagent beads. In addition, even when the reagent is formed in a substantially uniform size of beads through the lyophilizing process, the reagent may be exposed to humidity when the reagent beads are loaded into the reagent chamber 30 and the performance of the reagent may be degraded.

To solve these problems, a freezing process may be separated from a drying process. Hereinafter, a method of manufacturing a solid reagent according to an embodiment of the present inventive concept will be described in detail.

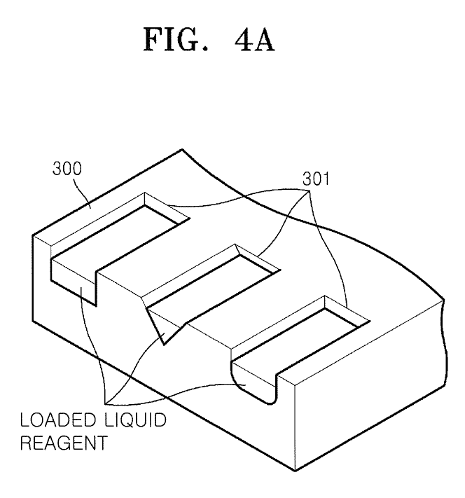

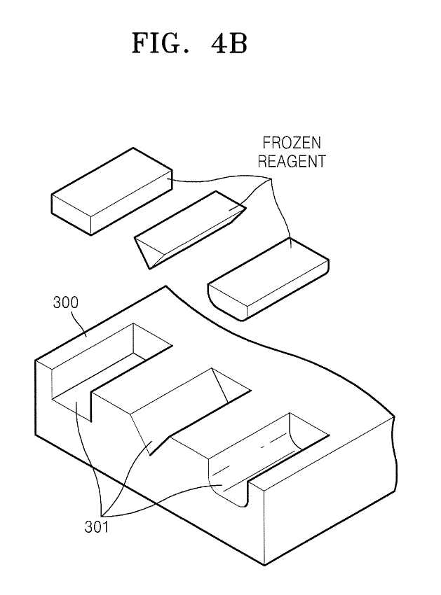

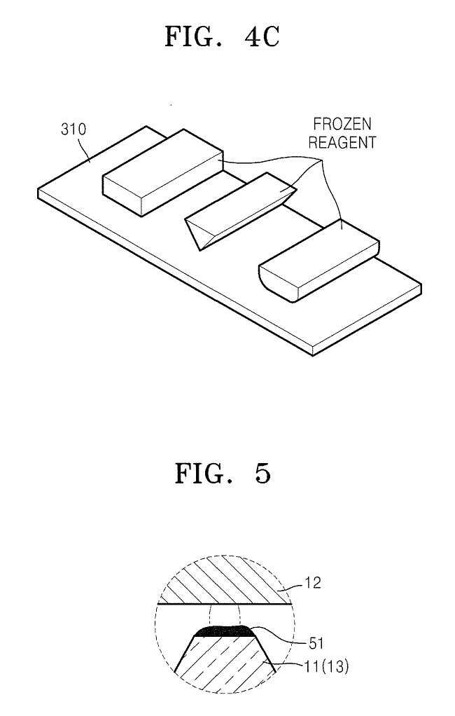

First, as illustrated in FIG. 4A, a mold 300 having a plurality of cavities 301 that receive a reagent is prepared. A liquid reagent is loaded to the reagent cavities 301 through a loading member such as a pipette. The liquid reagent can be loaded in an accurate amount and a degree of volume dispersion of the amount may be adjusted to be within 3% derivation. To reduce the volume of the reagent loaded, the concentration of the liquid reagent may be higher than that required to detect a material to be detected. The description “a reagent is loaded in an accurate amount” means that the reagent is loaded in a predetermined amount that is suitable or necessary to assay a sample and detect the target material to be detected in the sample, and therefore, any dilution or concentration is not necessary to carry out the assay. As an example, the reagent cavities 301 may have markings indicating a predetermined amount to help loading of an accurate amount of the liquid reagent.

For example, the mold 300 may be formed of PDMA and have a thickness of a few millimeters. However, the material for forming the mold 300 may not be limited to PDMA and can be any material that is chemically and biologically stable and flexible. The reagent cavities 301 may have the same shapes. In another embodiment of the present inventive concept, the reagent cavities 301 may have different shapes from each other. In the latter case, the reagent cavities 301 may contain different types of reagents.

The reagent for a blood test may be a reagent for detecting, for example, serum, aspartate aminotransferase (AST), albumin (ALB), alkaline phosphatase (ALP), alkaline aminotransferase (ALT), amylase (AMY), urea nitrogen (BUN), calcium (Ca⁺⁺), total cholesterol (CHOL), creatine kinase (CK), chloride (Cl⁻), creatinine (CREA), direct bilirubin (D-BIL), gamma glutamyl transferase (GGT), glucose (GLU), high-density lipoprotein cholesterol (HDL), potassium (K⁺), lactate dehydrogenase (LDH), low-density lipoprotein cholesterol (LDL), magnesium (Mg), phosphorus (PHOS), sodium (Na⁺), total carbon dioxide (TCO₂), total bilirubin (T-BIL), triglycerides (TRIG), uric acid (UA), albumin (ALB), or total protein (TP). In addition, the microfluidic device according to the current embodiment can also be used to assay, in addition to blood, various other samples, such as urine, that can be taken from a human body or other organism.

A filler may be added to the liquid reagent. When the filler is added, the reagent has a porous structure when lyophilized. Therefore, when a sample diluent including the sample and the diluent is loaded into the reagent chamber 30, the lyophilized reagent can be easily dissolved. For example, the filler may be selected from the group consisting of bovine serum albumin (BSA), polyethylene glycol (PEG), dextran, mannitol, polyalcohol, myo-inositol, citric acid, ethylene diamine tetraacetic acid disodium salt (EDTA2Na), and polyoxyethylene glycol dodecyl ether (e.g., BRIJ-35®). The filler may include one or at least two filters selected from the fillers according to the type of the reagent used.

A surfactant may be added to the liquid reagent. For example, the surfactant may be selected from the group consisting of polyoxyethylene, lauryl ether, octoxynol, polyethylene alkyl alcohol, nonylphenol polyethylene glycol ether, ethylene oxide, ethoxylated tridecyl alcohol, polyoxyethylene nonylphenyl ether phosphate sodium salt, and sodium dodecyl sulfate. The surfactant may include one or at least two surfactants selected from those surfactants according to the type of the reagent used.

The mold 300 containing the liquid reagent described above is loaded into a freezing device and frozen. Herein, the term ‘freezing’ refers to freezing moisture contained in the liquid reagent.

As illustrated in FIG. 4B, the frozen reagent is separated from the mold 300. In this case, the reagent is unlikely to be broken because it is in a frozen state, and remains in a lump form having an accurate amount.

The frozen reagent separated from the mold 300 is placed on, for example, a tray 310 illustrated in FIG. 4C, and the tray 310 is loaded into a lyophilizing device. Then a drying program for removing moisture is performed. The drying program may be appropriately selected according to the amount and/or kind of a reagent used. In most cases, the drying process uses a sublimating process whereby frozen water is directly changed into a vapor. However, the entire drying process does not necessarily require sublimation, that is, only a part of the drying process may require sublimation. To perform the sublimating process, the pressure in the drying process may be adjusted to be equal to or lower than the triple point of water (6 mbar or 4.6 Torr). However, there is no need to maintain a predetermined pressure. In the drying process, the temperature may be changed. For example, after the freezing process is completely performed, the temperature may be gradually increased.

As described above, according to the current embodiment, a reagent is frozen in a freezing process and the frozen reagent is separated from the mold 300 and thus, the resultant reagent is less fragile. Accordingly, after the drying process is completed, the solid reagent may retain its lump form. In addition, a plurality of solid reagents may be very easily mass-produced. Furthermore, since the reagent lump having an accurate amount can be loaded into the reagent chamber 30, it is very easy to load an accurate or precise amount of a reagent into a microfluidic device.

The solid reagent prepared as described above is placed in the reagent chamber 30 formed in the bottom plate 11, or in the reagent chamber 30 defined by the bottom plate 11 and the partitioning plate 13. Then the top plate 12 is coupled to the bottom plate 11 or the partitioning plate 13, thereby completing the manufacture of the microfluidic device according to an embodiment of the present inventive concept.

In some cases, a reagent may be comprised of components that degrade the activity of the reagent when the components are mixed and lypophilized. Examples of such a reagent include a reagent for detecting alkaline phosphatase (ALP), a reagent for detecting alkaline aminotransferase (ALT), a reagent for detecting high-density lipoprotein cholesterol (HDL), and a reagent for detecting low-density lipoprotein cholesterol (LDL). In biochemical reactions, when a substance functioning as a substrate and an enzyme co-exist, the titter of the reagent may be degraded. In such a case, a reagent including the substrate needs to be separated from a reagent including the enzyme. Specifically, in the case of the reagent for detecting ALP, p-nitrophenolphosphate (PNPP) functioning as a substrate is unstable when the pH is 10 or higher, and aminomethanpropanol (AMP) and diethanolamin (DEA) each functioning as a buffer that is necessary in a reaction system have a pH of 11-11.5. Therefore, the substrate needs to be separated from AMP and DEA and lyophilized.

In the case of the reagent for detecting AMY, NaCl is necessarily needed for a buffer and a substrate. However, NaCl has excellent deliquescent characteristics and is difficult to lyophilize. Even when NaCl is lyophilized, the lyophilized NaCl immediately absorbs humidity and the shape thereof is changed, and titer of the reagent may be degraded. Therefore, NaCl has to be separated from a substrate.

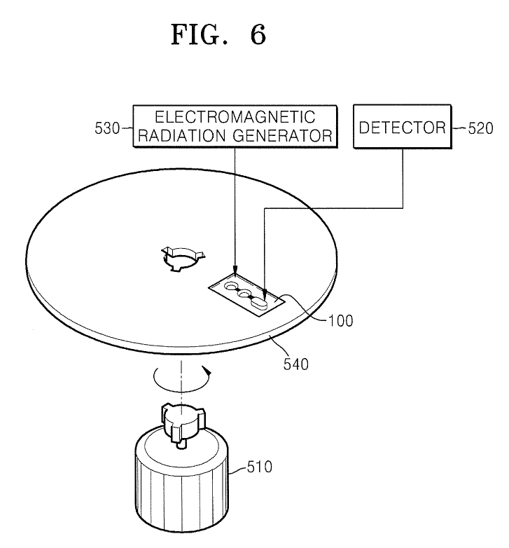

FIG. 6 is a schematic view of an analyzer using the microfluidic device 100 of FIG. 1. Referring to FIG. 6, a rotary driving unit 510 rotates the microfluidic device 100 so that a sample, a diluent, and a reagent therein are mixed by the effect of a centrifugal force. The rotary driving unit 510 moves the microfluidic device 100 to a predetermined position so that the reagent chamber 30 faces a detector 520. Although the rotary driving unit 510 is not entirely shown, the rotary driving unit 510 may further include a motor drive device (not shown) for controlling an angular position of the microfluidic device 100. The motor drive device may use a step motor or a direct-current motor. The detector 520 detects, for example, optical characteristics, such as fluorescent, luminescent, and/or absorbent characteristics, of a material to be detected. An electromagnetic radiation generator 530 is used to operate the valves 51 and 52, and emits, for example, a laser beam.

A method of analyzing the sample will now be described in detail. The diluent, such as a buffer or distilled water, is loaded into the diluent chamber 20 of the microfluidic device 100, and then, the sample, such as blood taken from a subject to be tested or serum separated from the blood, is loaded into the sample chamber 10.

Then, the microfluidic device 100 is installed in the analyzer illustrated in FIG. 6. If the microfluidic device 100 is chip-shaped, the microfluidic device 100 cannot be directly mounted on the rotary driving unit 510. In this case, the microfluidic device 100 is inserted into an adaptor 540 and the adaptor 540 is mounted on the rotary driving unit 510. In this regard, since a fluid flows from the sample chamber 10 to the reagent chamber 30, the microfluidic device 100 may be inserted in such a way that the sample chamber 10 is positioned closer to a rotary center of the adaptor 540 than the reagent chamber 30. The rotary driving unit 510 rotates the microfluidic device 100 so that the valve 51 faces the electromagnetic radiation generator 530. When electromagnetic radiation is irradiated to the valve 51, a material of the valve 51 melts due to electromagnetic radiation energy and a channel between the sample chamber 10 and the diluent chamber 20 is opened as illustrated in FIG. 5. The sample flows to the diluent chamber 20 by a centrifugal force. The rotary driving unit 510 shakes the microfluidic device 100 in a reciprocating motion to mix the sample with the diluent, thereby forming a sample diluent (i.e., a mixture of the sample and the diluent). Then, electromagnetic radiation is irradiated on the valve 52 to open a channel between the diluent chamber 20 and the reagent chamber 30 and the sample diluent is loaded into the reagent chamber 30. As a result, the solid reagent contained in the reagent chamber 30 is mixed with the sample diluent and melts. To dissolve the solid reagent by mixing with the sample diluent, the rotary driving unit 510 may shake the microfluidic device 100 in a reciprocating motion a few times. As a result, a reagent mixture is formed in the reagent chamber 30.

Then, the reagent chamber 30 is moved to face the detector 520 so as to identify whether a material to be detected is present in the reagent mixture in the reagent chamber 30, and to measure the amount of the material to be detected.

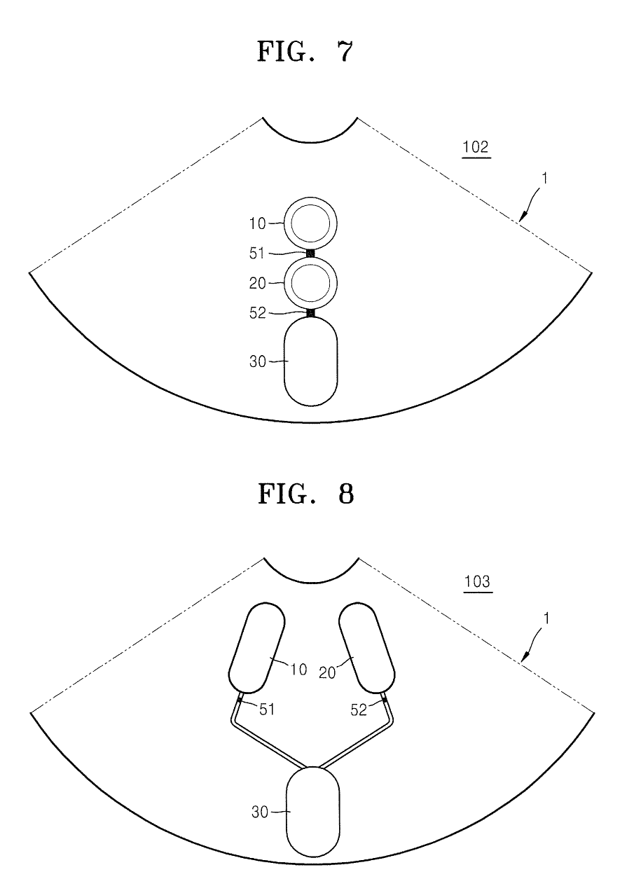

FIG. 7 is a plan view of a microfluidic device 102 according to another embodiment of the present inventive concept. Referring to FIG. 7, the microfluidic device 102 according to the current embodiment is disk-shaped and can be directly mounted on a rotary driving unit 510 of an analyzer. Although only a part of the microfluidic device 102 is illustrated in FIG. 7, the platform 1 may be circular and disk-shaped. The platform 1 may have the two-layer structure illustrated in FIG. 2 or the three-layer structure illustrated in FIG. 3.

The platform 1 includes a sample chamber 10, a diluent chamber 20, and a reagent chamber 30. The reagent chamber 30 may be located farther from the rotary center of the platform 1 than the sample chamber 10 and the diluent chamber 20. A valve 51 is formed between the sample chamber 10 and the diluent chamber 20 and a valve 52 is formed between the diluent chamber 20 and the reagent chamber 30. The reagent chamber 30 is to accommodate a solid reagent.

FIG. 8 is a plan view of an example of a modification of the microfluidic device 102 of FIG. 7. In a microfluidic device 103 illustrated in FIG. 8, a sample chamber 10 and a diluent chamber 20 are connected to a reagent chamber 30. A valve 51 is formed between the sample chamber 10 and the reagent chamber 30 and a valve 52 is formed between the diluent chamber 20 and the reagent chamber 30.

A method of analyzing a sample will now be described in detail. A liquid diluent, such as a buffer or distilled water, is loaded into the diluent chamber 20 of the microfluidic device 102 or 103. The sample is loaded into the sample chamber 10. Examples of the sample include blood taken from a subject to be tested and a serum separated from the blood.

Then, the microfluidic device 102 or 103 is mounted on the rotary driving unit 510 of the analyzer (see FIG. 6). The rotary driving unit 510 rotates the microfluidic device 102 or 103.

Then, the rotary driving unit 510 rotates in such a way that each of the valves 51 and 52 faces the electromagnetic radiation generator 530. When electromagnetic radiation is irradiated on the valves 51 and 52, a material forming the valve 51 and a material forming the valve 52 melt due to the electromagnetic radiation energy. When the microfluidic device 102 or 103 is rotated, the sample and the diluent are loaded into the reagent chamber 30 by a centrifugal force. The solid reagent contained in the reagent chamber 30 is mixed with the sample diluent that includes the sample and the diluent, and dissolved. Then, the reagent chamber 30 is moved to face the detector 520 to determine whether a target material to be detected is present in the reagent mixture in the reagent chamber 30, and the amount of the material to be detected.

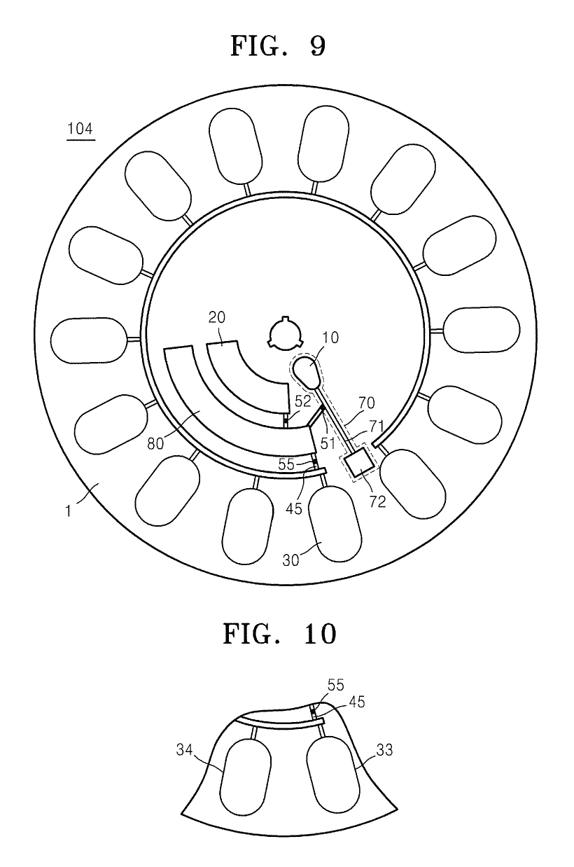

FIG. 9 is a plan view of a microfluidic device 104 according to another embodiment of the present inventive concept. Referring to FIG. 9, the microfluidic device 104 according to the current embodiment is disk-shaped, and can be directly mounted on the rotary driving unit 510 of the analyzer (see FIG. 6). The microfluidic device 104 includes a centrifuging unit 70 for isolating a supernatant from a sample. For example, when a sample such as whole blood is loaded, the centrifuging unit 70 separates the whole blood into serum and precipitations. A platform 1 may be disk-shaped. The platform 1 may have the two-layer structure illustrated in FIG. 2 or the three-layer structure illustrated in FIG. 3.

Hereinafter, a portion of the platform 1 located close to a center of the platform 1 will be referred to as an inner portion, and a portion of the platform 1 located far from the center will be referred to as an outer portion. The sample chamber 10 is closer to the center of the platform 1 than any other element that forms the microfluidic device 104. The centrifuging unit 70 includes a centrifuging portion 71 positioned radially outside the sample chamber 10 and a precipitations collector 72 positioned at an end of the centrifuging portion 71. When a sample is centrifuged, the supernatant remains in the sample chamber 10 or flows to the centrifuging portion 71, and heavy precipitations flow to the precipitations collector 72.

A diluent chamber 20 contains a diluent. The centrifuging portion 71 and the diluent chamber 20 are connected to a mixing chamber 80. A valve 51 is placed between the centrifuging portion 71 and the mixing chamber 80 and a valve 52 is placed between the diluted chamber 20 and the mixing chamber 80.

A plurality of reagent chambers 30 are positioned along a circumferential direction of the platform 1. The mixing chamber 80 is connected to the detection chambers 30 by a channel 45. The channel 45 includes a valve 55. The valve 55 may be formed of the same material as the valve 51 and the valve 52. Each of the detection chambers 30 accommodates a solid reagent.

A method of analyzing a sample will now be described in detail. A liquid diluent, such as a buffer or distilled water, is loaded into the diluent chamber 20 of the microfluidic device 104. A sample such as blood taken from a subject to be tested is loaded into the sample chamber 10.

Then, the microfluidic device 104 is mounted on the rotary driving unit 510 of the analyzer (see FIG. 6). The rotary driving unit 110 rotates the microfluidic device 104. Thus, due to the centrifugal force, the supernatant of the sample contained in the sample chamber 10 remains in the sample chamber 10 or flows to the centrifuging portion 71, and relatively heavy precipitations of the sample contained in the sample chamber 10 flow to the precipitations collector 72.

Then, the rotary driving unit 510 moves the microfluidic device 104 so that the valves 51 and 52 face the electromagnetic radiation generator 530. When electromagnetic radiation is irradiated to the valves 51 and 52, a valve forming material of the valves 51 and 52 melts due to electromagnetic radiation energy. When the microfluidic device 106 is rotated, the sample and the diluent are loaded into the mixing chamber 80, thereby forming a diluent sample including the sample and the diluent in the mixing chamber 80. To mix the sample with the diluent, the rotary driving unit 510 (as shown in FIG. 6) may laterally shake the microfluidic device 104 a few times.

Then, the rotary driving unit 510 (FIG. 6) moves the microfluidic device 104 so that the valve 55 faces the electromagnetic radiation generator 530 (FIG. 6). When electromagnetic radiation is irradiated to the valve 55, a valve forming material of the valve 55 melts due to the electromagnetic radiation energy and the channel 45 is opened. When the microfluidic device 104 rotates, the diluted sample is loaded into the reagent chamber 30 through the channel 45. The solid reagent is mixed with the diluent sample and melts. To dissolve the solid reagent, the rotary driving unit 510 may laterally shake the microfluidic device 104 a few times.

Then, the reagent chamber 30 is moved to face the detector 520 (FIG. 6) so as to identify whether a material to be detected is present in the reagent mixture in the reagent chamber 30, and to measure the amount of the detected material.

Hereinafter, a detection process including 2-step reactions, such as a process of detecting HDL from a sample, will be described with reference to the microfluidic device 104 illustrated in FIG. 9. In this case, as illustrated in FIG. 10, a first reagent chamber 33 accommodates a first reagent in a solid state, and a second reagent chamber 34 accommodates the first reagent in the solid state and a second reagent in a solid state. The first reagent and the second reagent have components as described below.

<First Solid Reagent>
PIPES (piperazine-1,4-bis(2-ethanesulfonic acid)): 30 MMOL/L
4-AAP (4-aminoantipyrine): 0.9 MMOL/L
POD (peroxidase): 240 U/L
ASOD (ascorbic oxidase): 2700 U/L
anti human b-lipoprotein antibody

<Second Solid Reagent>
PIPES (piperazine-1,4-bis(2-ethanesulfonic acid)): 30 MMOL/L
CHE (cholesterol esterase): 4000 U/L
COD (cholesterol oxidase): 20000 U/L
H-DASO (N-(2-hydroxy-3-sulfopropyl)-3,5-dimethoxyaniline): 0.8 MMOL/L

The first reagent is mixed in the first reagent chamber 33 with a diluted sample and kept there at about 37° C. for 5 minutes. As a result, HDL, LDL, very low density lipoprotein (VLDL), and chylomicron are formed into soluble HDL, and then soluble HDL is decomposed into cholesterol and hydrogen peroxide. The hydrogen peroxide is decomposed into water and oxygen.

The first reagent, the second reagent, and a diluted sample are mixed in the second reagent chamber 34 and kept there at about 37° C. for 5 minutes. As a result, HDL, LDL, VLDL, and chylomicron are formed into soluble HDL due to an enzyme reaction caused by the first reagent, and the soluble HDL is decomposed into cholestenone and hydrogen peroxide. The hydrogen peroxide is decomposed into water and oxygen. The residual HDL forms a pigment through an enzyme reaction with the second reagent. Absorbance of the first and second detection chambers 33 and 34 was measured by irradiating light thereon using the detector 520 (see FIG. 6).

Based on the two results of measuring the absorbance, it can be identified whether HDL is present and the amount of HDL can be calculated.

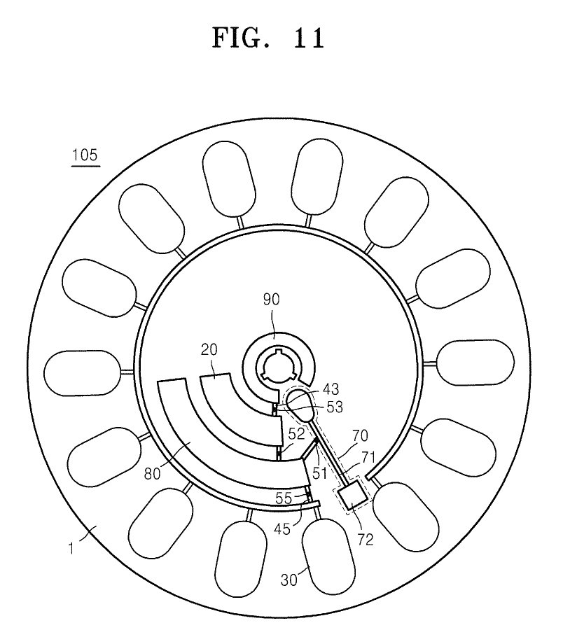

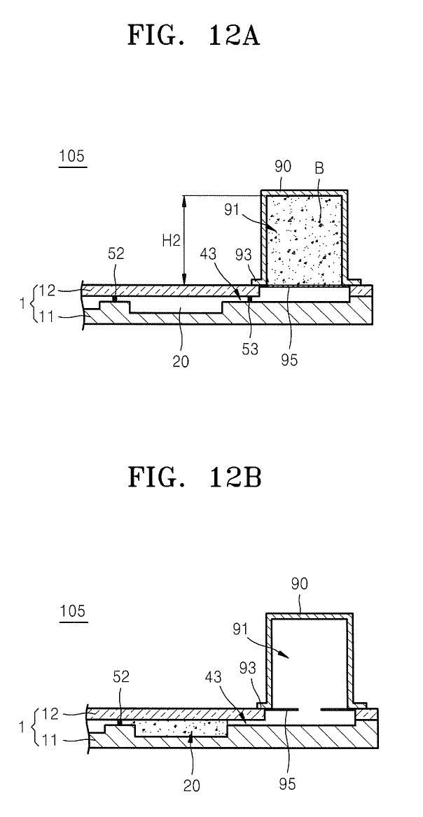

FIG. 1 is a plan view of a microfluidic device 105 according to another embodiment of the present inventive concept. FIGS. 12A and 12B are sectional views of the microfluidic device 105 of FIG. 11. The microfluidic device 105 according to the current embodiment is different from the microfluidic device 104 of FIG. 9 in that a container 90 containing a diluent is coupled to the platform 1 and the container 90 is connected to the diluent chamber 20 by a channel 43. The channel 43 may include a valve 53. The valve 53 may be formed of the same material as that forming the valves 51 and 52. However, in some embodiments, the channel 43 may not include the valve 53 because flow of the diluent is controlled by a lid 95.

Referring to FIGS. 11, 12A, and 12B, the platform 1 includes a top plate 12 and a bottom plate 11 coupled to the top plate 12. The container 90 includes a housing space 91 for housing a diluent. The container 90 may be formed by, for example, injection-molding a thermoplastic resin, and is fixed on the platform 1. The housing space 91 is sealed by the lid 95. The container 90 is turned upside down and the housing space 91 is filled with a diluent, and then the lid 95 is attached to an opening 93 of the container 90 so as to prevent leakage of the diluent. A fluid pouch that contains the diluent and is sealed but destroyable may be located inside the container 90.

The lid 95 is an example of a control member that controls the flow of the diluent from the container 90 to the channel 43. The lid 95 prevents leakage of the diluent contained in the housing space 91. The lid 95 may be destroyed or melted by electromagnetic radiation energy of, for example, a laser ray.

For example, the lid 95 may include a thin layer and an electromagnetic radiation absorption layer formed thereon. The thin layer may be formed of a metal. The electromagnetic radiation absorption layer may be formed by a coating of an electromagnetic radiation absorbing material. Due to the electromagnetic radiation absorption layer, the lid 95 absorbs external electromagnetic radiation and is destroyed or melted. The thin layer may be formed of, in addition to the metal, any material that can be destroyed or melted when exposed to electromagnetic radiation. In this regard, the thin layer may be formed of a polymer. A portion of the container 90 may be transparent so that externally projected electromagnetic radiation passes through the container 90 and reaches the lid 95.

The microfluidic device 105 is mounted on the rotary driving unit 510 of the analyzer (see FIG. 6), and electromagnetic radiation is projected on the lid 95 for a selected time period using the electromagnetic radiation generator 530 (see FIG. 6). As a result, as illustrated in FIG. 12B, the lid 95 is destroyed or melted.

Then, electromagnetic radiation is projected on the valve 53 using the electromagnetic radiation generator 530 (see FIG. 6). As a result, a material for forming the valve 53 melts and the channel 43 opens. The diluent contained in the housing space 91 flows to the diluent chamber 20 through the channel 43. Then, an analysis process is performed in the same manner as described with reference to the microfluidic device 104 of FIG. 9.

While aspects of the present invention have been particularly shown and described with reference to differing embodiments thereof, it should be understood that these exemplary embodiments should be considered in a descriptive sense only and not for purposes of limitation. Descriptions of features or aspects within each embodiment should typically be considered as available for other similar features or aspects in the remaining embodiments.

Thus, although a few embodiments have been shown and described, it would be appreciated by those of ordinary skill in the art that changes may be made in these embodiments without departing from the principles and spirit of the invention, the scope of which is defined in the claims and their equivalents.

What is claimed is:

1. A method of preparing a solid reagent, the method comprising:
providing a mold having a plurality of cavities (“reagent cavities”) to receive a liquid reagent;
loading the liquid reagent in the plurality of reagent cavities;
freezing the liquid reagent to obtain a frozen reagent;
separating the frozen reagent from the mold; and
drying the frozen reagent to remove moisture therefrom.

2. The method of claim 1, wherein the drying comprises sublimating moisture from the frozen reagent.

3. The method of claim 1, wherein the liquid reagent is concentrated to a level equal to or higher than the desired concentration, prior to being loaded into the plurality of reagent cavities.

4. The method of claim 1, wherein the plurality of reagent cavities accommodate at least two different liquid reagents.

5. The method of claim 1, wherein the plurality of reagent cavities have at least two different internal configurations.

6. The method of claim 1, wherein the mold is flexible.

7. The method of claim 1, wherein the solid reagent is selected from the group consisting of reagents for detecting serum, aspartate aminotransferase (AST), albumin (ALB), alkaline phosphatase (ALP), alkaline aminotransferase (ALT), amylase (AMY), urea nitrogen (BUN), calcium (Ca⁺⁺), total cholesterol (CHOL), creatine kinase (CK), chloride (Cl⁻), creatinine (CREA), direct bilirubin (D-BIL), gamma glutamyl transferase (GGT), glucose (GLU), high-density lipoprotein cholesterol (HDL), potassium (K⁺), lactate dehydrogenase (LDH), low-density lipoprotein cholesterol (LDL), magnesium (Mg), phosphorus (PHOS), sodium (Na⁺), total carbon dioxide (TCO₂), total bilirubin (T-BIL), triglycerides (TRIG), uric acid (UA), albumin (ALB), and total protein (TP).

8. The method of claim 1, wherein the liquid reagent comprises a filler.

9. The method of claim 8, wherein the filler is selected from the group consisting of bovine serum albumin (BSA), polyethylene glycol (PEG), dextran, mannitol, polyalcohol, myo-inositol, citric acid, ethylene diamine tetraacetic acid disodium salt (EDTA2Na), and polyoxyethylene glycol dodecyl ether (BRIJ-35).

10. The method of claim 1, wherein the solid reagent comprises a surfactant.

11. The method of claim 10, wherein the surfactant is selected from the group consisting of polyoxyethylene, lauryl ether, octoxynol, polyethylene alkyl alcohol, nonylphenol polyethylene glycol ether; ethylene oxide, ethoxylated tridecyl alcohol, polyoxyethylene nonylphenyl ether phosphate sodium salt, and sodium dodecyl sulfate.

12. A microfluidic device comprising:
a sample chamber to accommodate a sample to be examined;
a diluent chamber to accommodate a diluent;
a reagent chamber to accommodate a solid reagent; and
a channel connecting the sample chamber, the diluent chamber, and the reagent chamber.

13. The microfluidic device of claim 12, further comprising a valve controlling flow of a fluid through the channel.

14. The microfluidic device of claim 13, wherein the valve is formed of a valve forming material that melts by electromagnetic radiation energy.

15. The microfluidic device of claim 14, wherein the valve forming material is selected from a phase transition material and a thermoplastic resin, wherein the phase of the phase transition material or thermoplastic resin changes by electromagnetic radiation energy.

16. The microfluidic device of claim 15, wherein the valve forming material comprises micro heat-dissipating particles which are dispersed in the phase transition material, and absorb the electromagnetic radiation energy and dissipate the energy.

17. A microfluidic device comprising:
a platform having a plurality of chambers; and
a solid reagent accommodated in at least one of the plurality of chambers, wherein the solid reagent is used without an adjustment of its concentration prior to the use.

18. The microfluidic device of claim 17, wherein the plurality of chambers comprises:
a sample chamber to accommodate a sample to be examined;
a diluent chamber to accommodate a diluent; and
a plurality of reagent chambers to accommodate a solid reagent.

19. The microfluidic device of claim 18, wherein at least two different solid reagents are accommodated in the plurality of reagent chambers, wherein the different solid reagents have different shapes.

20. The microfluidic device of claim 17, further comprising:
a channel connecting the plurality of chambers; and
a valve placed in the channel to control flow of the fluid through the channel, wherein the valve blocks the channel when it is in a solid state and is melted by electromagnetic radiation energy to open the channel.
